SEQUENCE LISTING (8 sequences)

SEQ ID NO: 1 - DNA, 532 bp, Homo sapiens
gttgtcatcctgctctttgaatggaatctgttagaattctgtccgctgtgtcatacttggtgtgataaatgtatccattctcaataaaagctaagtctattcctggaatgttcccaaaatccctgtagatacgaaagtcagtatctgaaggaatgattccactctgaaaaacctcctgagccaccacagaagcaaaagggtgtttagctgctgaaacataagcttgaaccaaccaaggattttcaggacctgtttggaatacaagttctttccctcctacacctgctgcctctaggttaatgaatgcacgaatcaagctagcccaggggtgctgagtaatgaaaccatgactggcttgcaagacattttcctcagcaccattaaagagaaatatgacagcatgatgcaaggcttctgaagatgttgacaagacgcgaaggacttccagcatcactgagcagctaactgcatcatcactggcacctggtgagtttgctactgagtcaaaatgacaattagccaagacagcatg

SEQ ID NO: 2 - protein, 175 aa, Homo sapiens
HAVLANCHFDSVANSPGASDDAVSCSVMLEVLRVLSTSSEALHHAVIFLFNGAEENVLQASHGFITQHPWASLIRAFINLEAAGVGGKELVFQTGPENPWLVQAYVSAAKHPFASVVAQEVFQSGIIPSDTDFRIYRDFGNIPGIDLAFIENGYIYHTKYDTADRILTDSIQRAG

SEQ ID NO: 3 - DNA, 504 bp, Homo sapiens
cctgctctttgaatggaatctgttagaattctgtccgctgtgtcatacttggtgtgataaatgtatccattctcaataaaagctaagtctattcctggaatgttcccaaaatccctgtagatacgaaagtcagtatctgaaggaatgattccactctgaaaaacctcctgagccaccacagaagcaaaagggtgtttagctgctgaaacataagcttgaaccaaccaaggattttcaggacctgtttggaatacaagttctttccctcctacacctgctgcctctaggttaatgaatgcacgaatcaagctagcccaggggtgctgagtaatgaaaccatgactggcttgcaagacattttcctcagcaccattaaagagaaatatgacagcatgatgcaaggcttctgaagatgttgacaagacgcgaaggacttccagcatcactgagcagctaactgcatcatcactggcacctggtgagtttgctactgagtcaaatgac

SEQ ID NO: 4 - protein, 167 aa, Homo sapiens
SFDSVANSPGASDDAVSCSVMLEVLRVLSTSSEALHHAVIFLFNGAEENVLQASHGFITQHPWASLIRAFINLEAAGVGGKELVFQTGPENPWLVQAYVSAAKHPFASVVAQEVFQSGIIPSDTDFRIYRDFGNIPGIDLAFIENGYIYHTKYDTADRILTDSIQRA

SEQ ID NO: 5 - DNA, 478 bp, Homo sapiens
tgttaccaatctgaaggaatgattccactctgaaaaacctcctgagccaccacagaagcaaaagggtgtttagctgctgaaacataagcttgaaccaaccaaggattttcaggacctgtttggaatacaagttctttccctcctacacctgctgcctctaggttaatgaatgcacgaatcaagctagcccaggggtgctgagtaatgaaaccatgactggcttgcaagacattttcctcagcaccattaaagagaaatatgacagcatgatgcaaggcttctgaagatgttgacaagacgcgaaggacttccagcatcactgagcagctaactgcatcatcactggcacctggtgagtttgctactgagtcaaaatgacaattagccaagacagcatgctggggttcaatctcttggggtccagctttaccaccacattggtgaaggtggcataatagcctgtaaaccctcccagaaa

SEQ ID NO: 6 - protein, 157 aa, Homo sapiens
SGRVYRLLCHLHQCGGKAGPQEIEPQHAVLANCHFDSVANSPGASDDAVSCSVMLEVLRVLSTSSEALHHAVIFLFNGAEENVLQASHGFITQHPWASLIRAFINLEAAGVGGKELVFQTGPENPWLVQAYVSAAKHPFASVVAQEVFQSGIIPSDW

SEQ ID NO: 7 - DNA, 613 bp, Homo sapiens
gttgtcatcctgctctttgaatggaatctgttagaattctgtccgctgtgtcatacttggtgtgataaatgtatccattctcaataaaagctaagtctattcctggaatgttcccaaaatccctgtagatacgaaagtcaccaatctgaaggaatgattccactctgaaaaacctcctgagccaccacagaagcaaaagggtgtttagctgctgaaacataagcttgaaccaaccaaggattttcaggacctgtttggaatacaagttctttccctcctacacctgctgcctctaggttaatgaatgcacgaatcaagctagcccaggggtgctgagtaatgaaaccatgactggcttgcaagacattttcctcagcaccattaaagagaaatatgacagcatgatgcaaggcttctgaagatgttgacaagacgcgaaggacttccagcatcactgagcagctaactgcatcatcactggcacctggtgagtttgctactgagtcaaaatgacaattagccaagacagcatgctggggttcaatctcttggggtccagctttaccaccacattggtgaaggtggcataatagcctgtaaaccctcccagaaa

SEQ ID NO: 8 - protein, 203 aa, Homo sapiens
  misc_feature ()..(): X = any amino acid
SGRVYRLLCHLHQCGGKAGPQEIEPQHAVLANCHFDSVANSPGASDDAVSCSVMLEVLRVLSTSSEALHHAVIFLFNGAEENVLQASHGFITQHPWASLIRAFINLEAAGVGGKELVFQTGPENPWLVQAYVSAAKHPFASVVAQEVFQSGIIPSDWDFRIYRDFGNIPGIDLAFIENGYIYHTKYDTADRILTDSIQRAGXQ

(19) United States
(12) Patent Application Publication
(10) Pub. No.: US 2003/0049670 A1
(43) Pub. Date: Mar. 13, 2003

(54) Regulation of human leucine aminopeptidase-like enzyme

(76) Inventors: Shyam Ramakrishnan, Brighton, MA (US); Yonghong Xiao, Cambridge, MA (US)

Correspondence Address: BANNER & WITCOFF, 1001 G STREET N W, SUITE 1100, WASHINGTON, DC, 20001, US

(21) Appl. No.: 10/239,422

(22) PCT Filed: Mar. 23, 2001
(86) PCT No.: PCT/EP01/03331

(51) Int. Cl. C12Q 1/68; C07H 21/04; A61K 38/48; C12N 9/64; C12P 21/02; C12N 5/06; A61K 48/00

(52) U.S. Cl. 435/6; 435/69.1; 435/320.1; 435/325; 435/226; 536/23.2; 424/94.63; 514/44

(57) ABSTRACT

Reagents which regulate human leucine aminopeptidase-like enzyme activity and reagents which bind to human leucine aminopeptidase-like enzyme gene products can be used to regulate cellular and extracellular polypeptide degradation. Such regulation is particularly useful for treating cancer, HIV, autoimmune disease, and high blood pressure.

### TECHNICAL FIELD OF THE INVENTION

The invention relates to the area of regulation of cellular and extracellular degradation of polypeptides. More particularly, the invention relates to the regulation of human leucine aminopeptidase-like enzyme activity to increase or decrease cellular and extracellular polypeptide degradation.

### BACKGROUND OF THE INVENTION

Aminopeptidases comprise a family of zinc metalloproteinases that catalyze the removal of N-terminal residues from protein substrates. A leucine aminopeptidase catalyzes the release of an N-terminal amino acid wherein the N-terminal amino acid is preferably Leu, but can be other amino acids such as Pro. A leucine aminopeptidase can also hydrolyze amino acid amides and methyl esters. Elevated levels of leucine aminopeptidase is correlated with various cancers, autoimmune diseases, and HIV. Leucine aminopeptidase has also been shown to decrease high blood pressure.

### SUMMARY OF THE INVENTION

It is an object of the invention to provide reagents and methods of regulating cellular and extracellular degradation of polypeptides. These and other objects of the invention are provided by one or more of the embodiments described below.

One embodiment of the invention is a leucine aminopeptidase-like enzyme (LALE) polypeptide comprising an amino acid sequence selected from the group consisting of:

amino acid sequences which are at least about 50% identical to the amino acid sequence shown in SEQ ID NO: 2;

the amino acid sequence shown in SEQ ID NO: 2;

amino acid sequences which are at least about 50% identical to the amino acid sequence shown in SEQ ID NO: 4;

the amino acid sequence shown in SEQ ID NO: 4;

amino acid sequences which are at least about 50% identical to the amino acid sequence shown in SEQ ID NO: 6;

the amino acid sequence shown in SEQ ID NO: 6;

amino acid sequences which are at least about 50% identical to the amino acid sequence shown in SEQ ID NO: 8; and

the amino acid sequence shown in SEQ ID NO: 8.

Yet another embodiment of the invention is a method of screening for agents which decrease the activity of LALE. A test compound is contacted with a LALE polypeptide comprising an amino acid sequence selected from the group consisting of:

amino acid sequences which are at least about 50% identical to the amino acid sequence shown in SEQ ID NO: 2;

the amino acid sequence shown in SEQ ID NO: 2;

amino acid sequences which are at least about 50% identical to the amino acid sequence shown in SEQ ID NO: 4;

the amino acid sequence shown in SEQ ID NO: 4;

amino acid sequences which are at least about 50% identical to the amino acid sequence shown in SEQ ID NO: 6;

the amino acid sequence shown in SEQ ID NO: 6;

amino acid sequences which are at least about 50% identical to the amino acid sequence shown in SEQ ID NO: 8; and

the amino acid sequence shown in SEQ ID NO: 8.

Binding between the test compound and the LALE polypeptide is detected. A test compound which binds to the LALE polypeptide is thereby identified as a potential agent for decreasing the activity of LALE.

Another embodiment of the invention is a method of screening for agents which decrease the activity of LALE. A test compound is contacted with a polynucleotide encoding a LALE polypeptide, wherein the polynucleotide comprises a nucleotide sequence selected from the group consisting of:

nucleotide sequences which are at least about 50% identical to the nucleotide sequence shown in SEQ ID NO: 1;

the nucleotide sequence shown in SEQ ID NO: 1;

nucleotide sequences which are at least about 50% identical to the nucleotide sequence shown in SEQ ID NO: 3;

the nucleotide sequence shown in SEQ ID NO: 3;

nucleotide sequences which are at least about 50% identical to the nucleotide sequence shown in SEQ ID NO: 5;

the nucleotide sequence shown in SEQ ID NO: 5;

nucleotide sequences which are at least about 50% identical to the nucleotide sequence shown in SEQ ID) NO: 7; and

the nucleotide sequence shown in SEQ ID NO: 7.

Binding of the test compound to the polynucleotide is detected. A test compound which binds to the polynucleotide is identified as a potential agent for decreasing the activity of LALE. The agent can work by decreasing the amount of the LALE through interacting with the LALE mRNA.

Another embodiment of the invention is a method of screening for agents which regulate the activity of LALE. A test compound is contacted with a LALE polypeptide comprising an amino acid sequence selected from the group consisting of:

amino acid sequences which are at least about 50% identical to the amino acid sequence shown in SEQ ID NO: 2;

the amino acid sequence shown in SEQ ID NO: 2;

amino acid sequences which are at least about 50% identical to the amino acid sequence shown in SEQ ID NO: 4;

the amino acid sequence shown in SEQ ID NO: 4;

amino acid sequences which are at least about 50% identical to the amino acid sequence shown in SEQ ID NO: 6;

the amino acid sequence shown in SEQ ID NO: 6;

amino acid sequences which are at least about 50% identical to the amino acid sequence shown in SEQ ID NO: 8; and

the amino acid sequence shown in SEQ ID NO: 8.

A LALE activity of the polypeptide is detected. A test compound which increases LALE activity of the polypeptide relative to LALE activity in the absence of the test compound is thereby identified as a potential agent for increasing the activity of LALE. A test compound which decreases LALE activity of the polypeptide relative to LALE activity in the absence of the test compound is thereby identified as a potential agent for decreasing the activity of LALE.

Even another embodiment of the invention is a method of screening for agents which decrease the activity of LALE. A test compound is contacted with a LALE product of a polynucleotide which comprises a nucleotide sequence selected from the group consisting of:

nucleotide sequences which are at least about 50% identical to the nucleotide sequence shown in SEQ ID NO: 1;

the nucleotide sequence shown in SEQ ID NO: 1;

nucleotide sequences which are at least about 50% identical to the nucleotide sequence shown in SEQ ID NO: 3;

the nucleotide sequence shown in SEQ ID NO: 3;

nucleotide sequences which are at least about 50% identical to the nucleotide sequence shown in SEQ ID NO: 5;

the nucleotide sequence shown in SEQ ID NO: 5;

nucleotide sequences which are at least about 50% identical to the nucleotide sequence shown in SEQ ID NO: 7; and

the nucleotide sequence shown in SEQ ID NO: 7.

Binding of the test compound to the LALE product is detected. A test compound which binds to the LALE product is thereby identified as a potential agent for decreasing the activity of LALE.

Still another embodiment of the invention is a method of reducing the activity of LALE. A cell is contacted with a reagent which specifically binds to a polynucleotide encoding a LALE polypeptide or the product encoded by the polynucleotide, wherein the polynucleotide comprises a nucleotide sequence selected from the group consisting of:

nucleotide sequences which are at least about 50% identical to the nucleotide sequence shown in SEQ ID NO: 1;

the nucleotide sequence shown in SEQ ID NO: 1;

nucleotide sequences which are at least about 50% identical to the nucleotide sequence shown in SEQ ID NO: 3;

the nucleotide sequence shown in SEQ ID NO: 3;

nucleotide sequences which are at least about 50% identical to the nucleotide sequence shown in SEQ ID NO: 5;

the nucleotide sequence shown in SEQ ID NO: 5;

nucleotide sequences which are at least about 50% identical to the nucleotide sequence shown in SEQ ID NO: 7; and

the nucleotide sequence shown in SEQ ID NO: 7.

LALE activity in the cell is thereby decreased.

The invention thus provides reagents and methods for regulating cellular and extracellular polypeptide degradation. Such reagents and methods can be used inter alia, to suppress growth of malignant cells, to treat or prevent HIV, to treat or prevent autoimmune diseases such as systemic lupus erythematosus, and to treat or prevent high blood pressure.

### BRIEF DESCRIPTION OF THE DRAWINGS

FIG. 1 shows the DNA-sequence encoding a LALE polypeptide.

FIG. 2 shows the amino acid sequence deduced from the DNA-sequence of FIG. 1.

FIG. 3 shows the DNA-sequence encoding a LALE polypeptide.

FIG. 4 shows the amino acid sequence deduced from the DNA-sequence of FIG. 3.

FIG. 5 shows the DNA-sequence encoding a LALE polypeptide.

FIG. 6 shows the amino acid sequence deduced from the DNA-sequence of FIG. 5.

FIG. 7 shows the DNA-sequence encoding a LALE polypeptide.

FIG. 8 shows the amino acid sequence deduced from the DNA-sequence of FIG. 7.

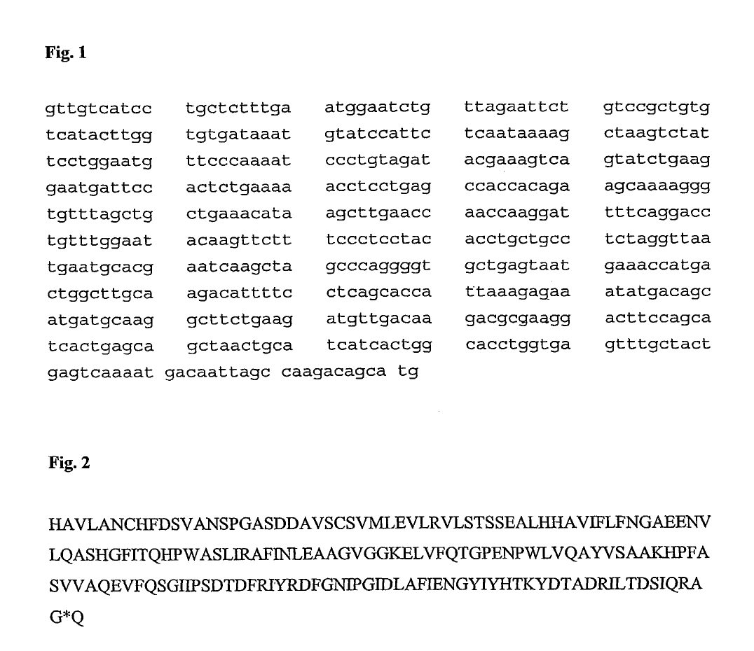

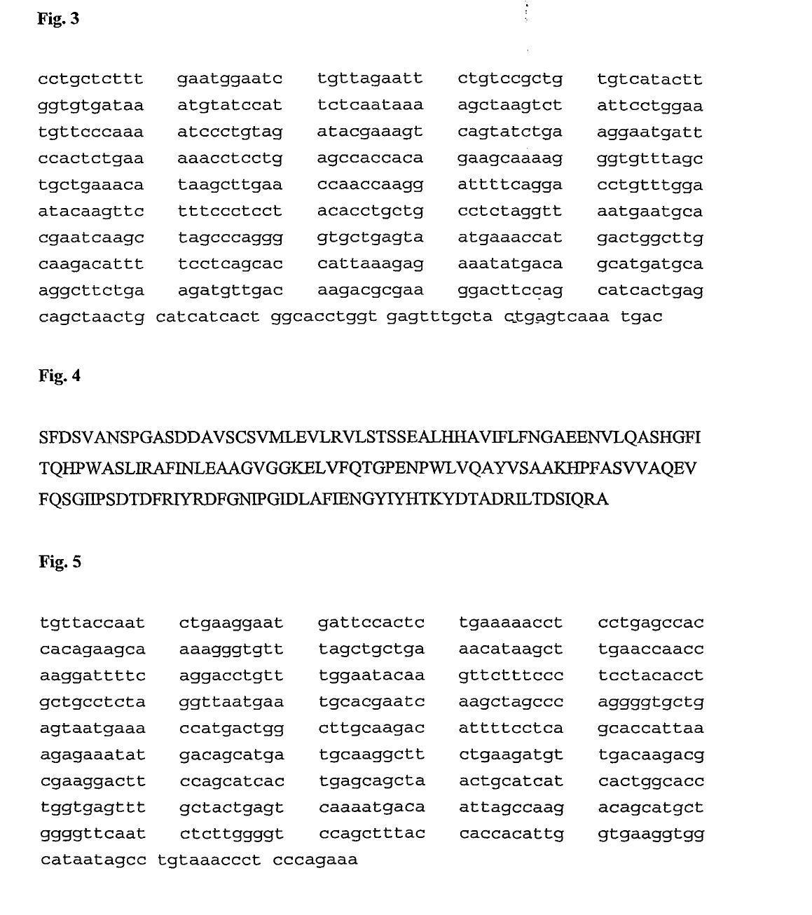

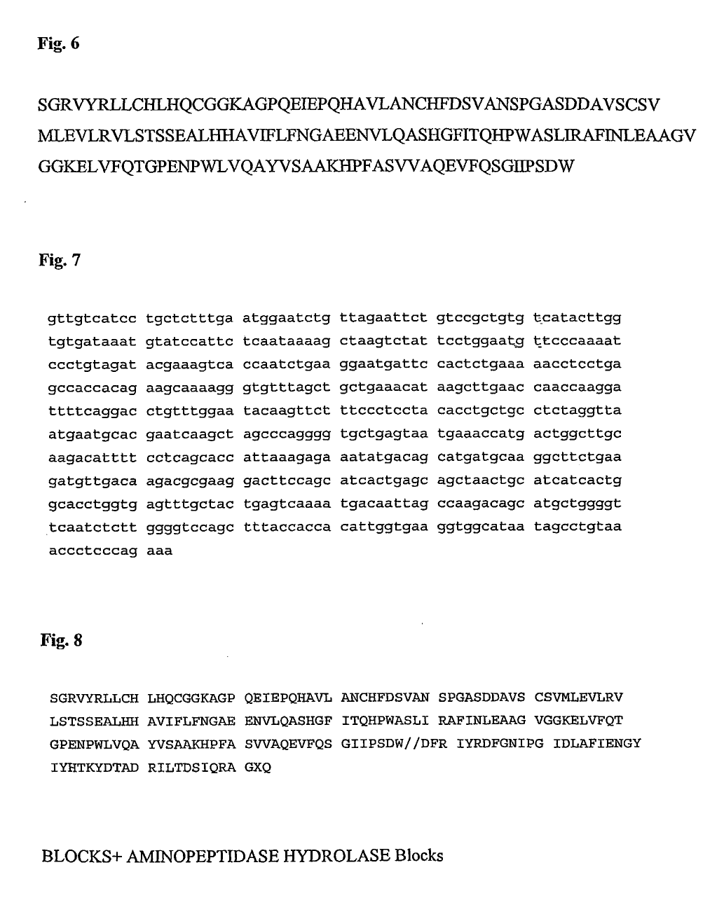

### DETAILED DESCRIPTION OF THE INVENTION

The invention relates to an isolated polynucleotide encoding a LALE polypeptide and being selected from the group consisting of:

a) a polynucleotide encoding a LALE polypeptide comprising an amino acid sequence selected from the group consisting of:

amino acid sequences which are at least about 50% identical to the amino acid sequence shown in SEQ ID NO: 2;

the amino acid sequence shown in SEQ ID NO: 2;

amino acid sequences which are at least about 50% identical to the amino acid sequence shown in SEQ ID NO: 4;

the amino acid sequence shown in SEQ ID NO: 4;

amino acid sequences which are at least about 50% identical to the amino acid sequence shown in SEQ ID NO: 6;

the amino acid sequence shown in SEQ ID NO: 6;

amino acid sequences which are at least about 50% identical to the amino acid sequence shown in SEQ ID NO: 8; and

the amino acid sequence shown in SEQ ID NO: 8.

b) a polynucleotide comprising the sequence of SEQ ID NOS: 1, 3, 5 or 7;

c) a polynucleotide which hybridizes under stringent conditions to a poly-nucleotide specified in (a) and (b);

d) a polynucleotide the sequence of which deviates from the polynucleotide sequences specified in (a) to (c) due to the degeneration of the genetic code; and

e) a polynucleotide which represents a fragment, derivative or allelic variation of a polynucleotide sequence specified in (a) to (d).

Furthermore, it has been discovered by the present applicant that activity of a leucine aminopeptidase-like enzyme (LALE), particularly a human LALE, can be modified to regulate degradation of cellular and extracellular polypeptides. Human LALE catalyzes the hydrolysis of a peptide bond by causing the release of an N-terminal amino acid where the N-terminal amino acid is preferably leucine, but can be other amino acids including proline (but not arginine or lysine). Amino acid amides and methyl esters are also hydrolyzed by LALE. Himmelhoch, Methods Ezymol. 19, 508, 1970; Delange & Smith, Leucine aminopeptidase and other N-terminal exopeptidases, in THE ENZYMES 81-118 (Boyer ed., 1971); van Loon- Klaasen et al. Biochem. Biophys. Res. Comm. 95, 334, 1980.

Human LALE can be used to develop treatments for various diseases, to develop diagnostic assays for these diseases, and to provide new tools for basic research especially in the fields of medicine and biology. Specifically, the present invention can be used to develop new drugs to treat or prevent cancer, HIV, and autoimmune diseases, such as systemic lupus erythematosus, multiple sclerosis, immune-mediated or type 1 diabetes mellitus, Crohn's disease, ulcerative colitis, psoriasis, scleroderma, Hashimoto's thyroiditis, and Grave's disease, and to treat or prevent high blood pressure.

Human Leucine Aminopeptidase-like Enzyme Polypeptides

Human LALE polypeptides according to the invention comprise an amino acid sequence as shown in SEQ ID NOS: 2, 4, or 6 or a portion of one of those amino acid sequences, or a biologically active variant of an amino acid sequence shown in SEQ ID NOS: 2, 4, or 6 as defined below. SEQ ID NOS: 1, 3, and 5 are complementary to the nucleotide sequences which encode SEQ ID NOS: 2, 4, and 6, respectively. Thus, a LALE polypeptide can be a portion of a leucine aminopeptidase-like enzyme molecule, a full-length LALE molecule, or a fusion protein comprising all or a portion of a LALE molecule. Most preferably, a LALE polypeptide has a leucine aminopeptidase activity. Leucine aminopeptidase activity can be measured, inter alia, as described in Example 2.

Biologically Active Variants

LALE variants which are biologically active, i.e., retain a leucine aminopeptidase activity, also are LALE polypeptides. Preferably, naturally or non-naturally occurring LALE variants have amino acid sequences which are at least about 50, preferably about 75, 90, 96, or 98% identical to an amino acid sequence shown in SEQ ID NOS: 2, 4, or 6. Percent identity between a putative LALE variant and an amino acid sequence of SEQ ID NOS: 2, 4, or 6 is determined using the Blast2 alignment program.

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of a LALE polypeptide can be found using computer programs well known in the art, such as DNASTAR software. Whether an amino acid change results in a biologically active LALE polypeptide can readily be determined by assaying for LALE activity, as described, for example, in Example 2.

Fusion Proteins

Fusion proteins can comprise at least 5, 6, 8, 10, 25, or 50 or more contiguous amino acids of an amino acid sequence shown in SEQ ID NOS: 2, 4, or 6. Fusion proteins are useful for generating antibodies against LALE amino acid sequences and for use in various assay systems. For example, fusion proteins can be used to identify proteins which interact with portions of a LALE polypeptide. Protein affinity chromatography or library- based assays for protein-protein interactions, such as the yeast two-hybrid or phage display systems, can be used for this purpose. Such methods are well known in the art and also can be used as drug screens.

A LALE fusion protein comprises two protein segments fused together by means of a peptide bond. The first protein segment comprises at least 5, 6, 8, 10, 25, or 50 or more contiguous amino acids of a LALE polypeptide. Contiguous amino acids for use in a fusion protein can be selected from the amino acid sequence shown in SEQ ID NOS: 2, 4, or 6 or from a biologically active variant of those sequences, such as those described above. The first protein segment also can comprise full-length LALE.

The second protein segment can be a full-length protein or a protein fragment or polypeptide. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags are used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP6 protein fusions. A fusion protein also can be engineered to contain a cleavage site located between the LALE polypeptide-encoding sequence and the heterologous protein sequence, so that the LALE polypeptide can be cleaved and purified away from the heterologous moiety.

A fusion protein can be synthesized chemically, as is known in the art. Preferably, a fusion protein is produced by covalently linking two protein segments or by standard procedures in the art of molecular biology. Recombinant DNA methods can be used to prepare fusion proteins, for example, by making a DNA construct which comprises coding sequences selected from the complements of SEQ ID NOS: 1, 3, or 5 in proper reading frame with nucleotides encoding the second protein segment and expressing the DNA construct in a host cell, as is known in the art. Many kits for constructing fusion proteins are available from companies such as Promega Corporation (Madison, Wis.), Stratagene (La Jolla, Calif.), CLONTECH (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Identification of Species Homologs

Species homologs of human LALE can be obtained using LALE polynucleotides (described below) to make suitable probes or primers for screening cDNA expression libraries from other species, such as mice, monkeys, or yeast, identifying cDNAs which encode homologs of LALE, and expressing the cDNAs as is known in the art.

LALE Polynucleotides

A LALE polynucleotide can be single- or double-stranded and comprises a coding sequence or the complement of a coding sequence for a LALE polypeptide. The complements of partial nucleotide sequences encoding LALE polypeptides are shown in SEQ ID NOS: 1, 3, and 5.

Degenerate nucleotide sequences encoding human LALE polypeptides, as well as homologous nucleotide sequences which are at least about 50, preferably about 75, 90, 96, or 98% identical to the complements of the nucleotide sequences shown in SEQ ID NOS: 1, 3, or 5 also are LALE polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of LALE polynucleotides which encode biologically active LALE polypeptides also are LALE polynucleotides.

Identification of Variants and Homologs of LALE Polynucleotides

Variants and homologs of the LALE polynucleotides described above also are LALE polynucleotides. Typically, homologous LALE polynucleotide sequences can be identified by hybridization of candidate polynucleotides to known LALE polynucleotides under stringent conditions, as is known in the art. For example, using the following wash conditions—2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each—homologous sequences can be identified which contain at most about 25-30% basepair mismatches. More preferably, homologous nucleic acid strands contain 15-25% basepair mismatches, even more preferably 5-15% basepair mismatches.

Species homologs of the LALE polynucleotides disclosed herein also can be identified by making suitable probes or primers and screening cDNA expression libraries from other species, such as mice, monkeys, or yeast. Human variants of LALE polynucleotides can be identified, for example, by screening human cDNA expression libraries. It is well known that the Tₘ of a double-stranded DNA decreases by 1-1.5° C. with every 1% decrease in homology (Bonner et al., J. Mol. Biol. 81, 123, 1973). Variants of human LALE polynucleotides or LALE polynucleotides of other species can therefore be identified by hybridizing a putative homologous LALE polynucleotide with a polynucleotide having a nucleotide sequence of SEQ ID NOS: 1, 3, or 5 or the complement thereof to form a test hybrid. The melting temperature of the test hybrid is compared with the melting temperature of a hybrid comprising LALE polynucleotides having perfectly complementary nucleotide sequences, and the number or percent of basepair mismatches within the test hybrid is calculated.

Nucleotide sequences which hybridize to LALE polynucleotides or their complements following stringent hybridization and/or wash conditions also are LALE polynucleotides. Stringent wash conditions are well known and understood in the art and are disclosed, for example, in Sambrook et al., MOLECULAR CLONING: A LABORATORY MANUAL, 2d ed., 1989, at pages 9.50-9.51.

Typically, for stringent hybridization conditions a combination of temperature and salt concentration should be chosen that is approximately 12-20° C. below the calculated Tₘ of the hybrid under study. The Tₘ of a hybrid between a LALE polynucleotide having a nucleotide sequence shown in SEQ ID NOS: 1, 3, 5 or 7 or the complement thereof and a polynucleotide sequence which is at least about 50, preferably about 75, 90, 96, or 98% identical to one of those nucleotide sequences can be calculated, for example, using the equation of Bolton and McCarthy, Proc. Natl. Acad Sci. U.S.A. 48, 1390 (1962):

Tₘ=81.5° C.−16.6(log₁₀[Na⁺])+0.41(% G+C)−0.63(% formamide)−600/l),

where l=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C.

Preparation of LALE Polynucleotides

A naturally occurring LALE polynucleotide can be isolated free of other cellular components such as membrane components, proteins, and lipids. Polynucleotides can be made by a cell and isolated using standard nucleic acid purification techniques, or synthesized using an amplification technique, such as the polymerase chain reaction (PCR), or by using an automatic synthesizer. Methods for isolating polynucleotides are routine and are known in the art. Any such technique for obtaining a polynucleotide can be used to obtain isolated LALE polynucleotides. For example, restriction enzymes and probes can be used to isolate polynucleotide fragments which comprise LALE nucleotide sequences. Isolated polynucleotides are in preparations which are free or at least 70, 80, or 90% free of other molecules.

LALE cDNA molecules can be made with standard molecular biology techniques, using LALE mRNA as a template. LALE cDNA molecules can thereafter be replicated using molecular biology techniques known in the art and disclosed in manuals such as Sambrook et al. (1989). An amplification technique, such as PCR, can be used to obtain additional copies of LALE polynucleotides using either human genomic DNA or cDNA as a template.

Alternatively, synthetic chemistry techniques can be used to synthesize LALE polynucleotides. The degeneracy of the genetic code allows alternate nucleotide sequences to be synthesized which will encode a LALE polypeptide having, for example, an amino acid sequence shown in SEQ ID NOS: 2, 4, 6 or 8 or a biologically active variant of one of those sequences.

Obtaining Full-length LALE Polynucleotides

The partial sequences of SEQ ID NOS: 1, 3, or 5 or their complements can be used to identify the corresponding fill length gene(s) from which they were derived. The partial sequences can be nick-translated or end-labeled with ³²P using polynucleotide kinase using labeling methods known to those with skill in the art (BASIC METHODS IN MOLECULAR BIOLOGY, Davis et al., eds., Elsevier Press, N.Y., 1986). For example, a lambda library prepared from human tissue can be screened directly with the labeled sequences of interest or the library can be converted en masse to pBluescript (Stratagene Cloning Systems, La Jolla, Calif. 92037) to facilitate bacterial colony screening (see Sambrook et al., 1989, pg. 1.20).

Both methods are well known in the art. Briefly, filters with bacterial colonies containing the library in pBluescript or bacterial lawns containing lambda plaques are denatured, and the DNA is fixed to the filters. The filters are hybridized with the labeled probe using hybridization conditions described by Davis et al., 1986. The partial sequences, cloned into lambda or pBluescript, can be used as positive controls to assess background binding and to adjust the hybridization and washing stringencies necessary for accurate clone identification. The resulting auto-radiograms are compared to duplicate plates of colonies or plaques; each exposed spot corresponds to a positive colony or plaque. The colonies or plaques are selected and expanded, and the DNA is isolated from the colonies for further analysis and sequencing.

Positive cDNA clones are analyzed to determine the amount of additional sequence they contain using PCR with one primer from the partial sequence and the other primer from the vector. Clones with a larger vector-insert PCR product than the original partial sequence are analyzed by restriction digestion and DNA sequencing to determine whether they contain an insert of the same size or similar as the mRNA size determined from Northern blot Analysis.

Once one or more overlapping cDNA clones are identified, the complete sequence of the clones can be determined, for example after exonuclease III digestion (McCombie et al., Methods 3, 33-40, 1991). A series of deletion clones are generated, each of which is sequenced. The resulting overlapping sequences are assembled into a single contiguous sequence of high redundancy (usually three to five overlapping sequences at each nucleotide position), resulting in a highly accurate final sequence.

Various PCR-based methods can be used to extend the nucleic acid sequences encoding the disclosed portions of human LALE to detect upstream sequences such as promoters and regulatory elements. For example, restriction-site PCR uses universal primers to retrieve unknown sequence adjacent to a known locus (Sarkar, PCR Methods Applic. 2, 318-322, 1993). Genomic DNA is first amplified in the presence of a primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR also can be used to amplify or extend sequences using divergent primers based on a known region (Triglia et al., Nucleic Acids Res. 16, 8186, 1988). Primers can be designed using commercially available software, such as OLIGO 4.06 Primer Analysis software (National Biosciences Inc., Plymouth, Minn.), to be 22-30 nucleotides in length, to have a GC content of 50% or more, and to anneal to the target sequence at temperatures about 68°-72° C. The method uses several restriction enzymes to generate a suitable fragment in the known region of a gene. The fragment is then circularized by intramolecular ligation and used as a PCR template.

Another method which can be used is capture PCR, which involves PCR amplification of DNA fragments adjacent to a known sequence in human and yeast artificial chromosome DNA (Lagerstrom et al., PCR Methods Applic. 1, 111-119, 1991). In this method, multiple restriction enzyme digestions and ligations also can be used to place an engineered double-stranded sequence into an unknown fragment of the DNA molecule before performing PCR.

Another method which can be used to retrieve unknown sequences is that of Parker et al., Nucleic Acids Res. 19, 3055-3060, 1991). Additionally, PCR, nested primers, and PROMOTERFINDER libraries (CLONTECH, Palo Alto, Calif.) can be used to walk genomic DNA. This process avoids the need to screen libraries and is useful in finding intron/exon junctions.

When screening for full-length cDNAs, it is preferable to use libraries that have been size-selected to include larger cDNAs. Randomly-primed libraries are preferable, in that they will contain more sequences which contain the 5′ regions of genes. Use of a randomly primed library may be especially preferable for situations in which an oligo d(T) library does not yield a full-length cDNA. Genomic libraries can be useful for extension of sequence into 5′ non-transcribed regulatory regions.

Commercially available capillary electrophoresis systems can be used to analyze the size or confirm the nucleotide sequence of PCR or sequencing products. For example, capillary sequencing can employ flowable polymers for electrophoretic separation, four different fluorescent dyes (one for each nucleotide) which are laser activated, and detection of the emitted wavelengths by a charge coupled device camera. Output/light intensity can be converted to electrical signal using appropriate software (e.g. GENOTYPER and Sequence NAVIGATOR, Perkin Elmer), and the entire process from loading of samples to computer analysis and electronic data display can be computer controlled. Capillary electrophoresis is especially preferable for the sequencing of small pieces of DNA which might be present in limited amounts in a particular sample.

Obtaining LALE Polypeptides

LALE polypeptides can be obtained, for example, by purification from human serum or cells, by expression of LALE polynucleotides, or by direct chemical synthesis.

Protein Purification

LALE polypeptides can be purified, for example, from human serum or from cells such as germinal B cells, parathyroid tumor cells, melanocytes, fetal heart cells, and cells of the pregnant uterus. A purified LALE polypeptide is separated from other compounds which normally associate with the LALE polypeptide, such as certain proteins, carbohydrates, or lipids, using methods well-known in the art. Such methods include, but are not limited to, size exclusion chromatography, ammonium sulfate fractionation, ion exchange chromatography, affinity chromatography, and preparative gel electrophoresis. A preparation of purified LALE polypeptides is at least 80% pure; preferably, the preparations are 90%, 95%, or 99% pure. Purity of the preparations can be assessed by any means known in the art, such as SDS-polyacrylamide gel electrophoresis. Enzymatic activity of the purified preparations can be assayed, for example, as described in Example 2.

Expression of LALE Polynucleotides

To express a LALE polypeptide, a LALE polynucleotide can be inserted into an expression vector which contains the necessary elements for the transcription and translation of the inserted coding sequence. Methods which are well known to those skilled in the art can be used to construct expression vectors containing sequences encoding LALE polypeptides and appropriate transcriptional and translational control elements. These methods include in vitro recombinant DNA techniques, synthetic techniques, and in vivo genetic recombination. Such techniques are described, for example, in Sambrook et al. (1989) and in Ausubel et al., CURRENT PROTOCOLS IN MOLECULAR BIOLOGY, John Wiley & Sons, New York, N.Y, 1989.

A variety of expression vector/host systems can be utilized to contain and express sequences encoding a LALE polypeptide. These include, but are not limited to, microorganisms, such as bacteria transformed with recombinant bacteriophage, plasmid, or cosmid DNA expression vectors; yeast transformed with yeast expression vectors, insect cell systems infected with virus expression vectors (e.g., baculovirus), plant cell systems transformed with virus expression vectors (e.g., cauliflower mosaic virus, CaMV; tobacco mosaic virus, TMV) or with bacterial expression vectors (e.g., Ti or pBR322 plasmids), or animal cell systems.

The control elements or regulatory sequences are those non-translated regions of the vector—enhancers, promoters, 5′ and 3′ untranslated regions—which interact with host cellular proteins to carry out transcription and translation. Such elements can vary in their strength and specificity. Depending on the vector system and host, any number of suitable transcription and translation elements, including constitutive and inducible promoters, can be used. For example, when cloning in bacterial systems, inducible promoters such as the hybrid lacZ promoter of the BLUESCRIPT phagemid (Stratagene, Lajolla, Calif.) or pSPORT1 plasmid (Life Technologies) and the like can be used. The baculovirus polyhedrin promoter can be used in insect cells. Promoters or enhancers derived from the genomes of plant cells (e.g., heat shock, RUBISCO, and storage protein genes) or from plant viruses (e.g., viral promoters or leader sequences) can be cloned into the vector. In mammalian cell systems, promoters from mammalian genes or from mammalian viruses are preferable. If it is necessary to generate a cell line that contains multiple copies of a nucleotide sequence encoding a LALE polypeptide, vectors based on SV40 or EBV can be used with an appropriate selectable marker.

Bacterial and Yeast Expression Systems

In bacterial systems, a number of expression vectors can be selected depending upon the use intended for a LALE polypeptide. For example, when a large quantity of a LALE polypeptide is needed for the induction of antibodies, vectors which direct high level expression of fusion proteins that are readily purified can be used. Such vectors include, but are not limited to, multifunctional E. coli cloning and expression vectors such as BLUESCRIPT (Stratagene). In a BLUESCRIPT vector, a sequence encoding a LALE polypeptide can be ligated in frame with sequences for the amino-terminal Met and the subsequent 7 residues of β-galactosidase so that a hybrid protein is produced. pIN vectors (Van Heeke & Schuster, J. Biol Chem. 264, 5503-5509, 1989 or pGEX vectors (Promega, Madison, Wis.) also can be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems can be designed to include heparin, thrombin, or factor Xa protease cleavage sites so that the cloned polypeptide of interest can be released from the GST moiety at will.

In the yeast Saccharomyces cerevisiae, a number of vectors containing constitutive or inducible promoters, such as alpha factor, alcohol oxidase, and PGH, can be used. For reviews, see Ausubel et al. (1989) and Grant et al., Methods Enzymol. 153, 516-544, 1987.

Plant and Insect Expression Systems

If plant expression vectors are used, the expression of sequences encoding LALE polypeptides can be driven by any of a number of promoters. For example, viral promoters such as the 35S and 19S promoters of CaMV can be used alone or in combination with the omega leader sequence from TMV (Takamatsu, EMBO J. 6, 307-311, 1987). Alternatively, plant promoters such as the small subunit of RUBISCO or heat shock promoters can be used (Coruzzi et al., EMBO J. 3, 1671-1680, 1984; Broglie et al., Science 224, 838-843, 1984; Winter et al., Results Probl Cell Differ. 17, 85-105, 1991). These constructs can be introduced into plant cells by direct DNA transformation or by pathogen-mediated transfection. Such techniques are described in a number of generally available reviews (e.g., Hobbs or Murry, in MCGRAW HILL YEARBOOK OF SCIENCE AND TECHNOLOGY, McGraw Hill, New York, N.Y., pp. 191-196, 1992).

An insect system also can be used to express a LALE polypeptide. For example, in one such system Autographa californica nuclear polyhedrosis virus (AcNPV) is used as a vector to express foreign genes in Spodoptera frugiperda cells or in Trichoplusia larvae. Sequences encoding LALE polypeptides can be cloned into a non-essential region of the virus, such as the polyhedrin gene, and placed under control of the polyhedrin promoter. Successful insertion of LALE polypeptides will render the polyhedrin gene inactive and produce recombinant virus lacking coat protein. The recombinant viruses can then be used to infect S. frugiperda cells or Trichoplusia larvae in which LALE polypeptides can be expressed (Engelhard et al., Proc. Nat. Acad. Sci. 91, 3224-3227, 1994).

Mammalian Expression Systems

A number of viral-based expression systems can be used to express LALE polypeptides in mammalian host cells. For example, if an adenovirus is used as an expression vector, sequences encoding LALE polypeptides can be ligated into an adenovirus transcription/translation complex comprising the late promoter and tripartite leader sequence. Insertion in a non-essential E1 nor E3 region of the viral genome can be used to obtain a viable virus which is capable of expressing a LALE polypeptide in infected host cells (Logan & Shenk, Proc. Natl. Acad. Sci. 81, 3655-3659, 1984). If desired, transcription enhancers such as the Rous sarcoma virus (RSV) enhancer can be used to increase expression in mammalian host cells.

Human artificial chromosomes (HACs) also can be used to deliver larger fragments of DNA than can be contained and expressed in a plasmid. HACs of 6M to 10M are constructed and delivered to cells via conventional delivery methods (e.g., liposomes, polycationic amino polymers, or vesicles).

Specific initiation signals also can be used to achieve more efficient translation of sequences encoding LALE polypeptides. Such signals include the ATG initiation codon and adjacent sequences. In cases where sequences encoding a LALE polypeptide, its initiation codon, and upstream sequences are inserted into the appropriate expression vector, no additional transcriptional or translational control signals may be needed. However, in cases where only coding sequence, or a fragment thereof, is inserted, exogenous translational control signals (including the ATG initiation codon) should be provided. The initiation codon should be in the correct reading frame to ensure translation of the entire insert. Exogenous translational elements and initiation codons can be of various origins, both natural and synthetic. The efficiency of expression can be enhanced by the inclusion of enhancers which are appropriate for the particular cell system which is used (see Scharf et al., Results Probl. Cell Differ. 20,125-162, 1994).

Host Cells

A host cell strain can be chosen for its ability to modulate the expression of the inserted sequences or to process the expressed LALE polypeptide in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding, and/or function. Different host cells which have specific cellular machinery and characteristic mechanisms for post-translational activities (e.g., CHO, HeLa, MDCK, HEK293, and WI38), are available from the American Type Culture Collection (ATCC; 10801University Boulevard, Manassas, Va. 20110-2209) and can be chosen to ensure the correct modification and processing of the foreign protein.

Stable expression is preferred for long-term, high-yield production of recombinant proteins. For example, cell lines which stably express LALE polypeptides can be transformed using expression vectors which can contain viral origins of replication and/or endogenous expression elements and a selectable marker gene on the same or on a separate vector. Following the introduction of the vector, cells can be allowed to grow for 1-2 days in an enriched medium before they are switched to a selective medium. The purpose of the selectable marker is to confer resistance to selection, and its presence allows growth and recovery of cells which successfully express the introduced LALE sequences. Resistant clones of stably transformed cells can be proliferated using tissue culture techniques appropriate to the cell type. See, for example, ANIMAL CELL CULTURE, R. I. Freshney, ed., 1986.

Any number of selection systems can be used to recover transformed cell lines. These include, but are not limited to, the herpes simplex virus thymidine kinase (Wigler et al., Cell 11, 223-32, 1977) and adenine phosphoribosyltransferase (Lowy et al., Cell 22, 817-23, 1980) genes which can be employed in tk⁻ or aprt⁻ cells, respectively. Also, antimetabolite, antibiotic, or herbicide resistance can be used as the basis for selection. For example, dhrf confers resistance to methotrexate (Wigler et al., Proc. Natl. Acad. Sci. 77, 3567-70, 1980) npt confers resistance to the aminoglycosides neomycin and G-418 (Colbere-Garapin et al., J. Mol. Biol. 150, 1-14, 1981), and als and pat confer resistance to chlorsulfuron and phosphinotricin acetyltransferase, respectively (Murry, 1992, supra). Additional selectable genes have been described. For example, trpB, allows cells to utilize indole in place of tryptophan; hisD, allows cells to utilize histinol in place of histidine (Hartman & Mulligan, Proc. Natl. Acad. Sci. 85, 8047-51, 1988). Visible markers such as antho-cyanins, β-glucuronidase and its substrate GUS, and luciferase and its substrate luciferin, can be used to identify transformants and to quantify the amount of transient or stable protein expression attributable to a specific vector system (Rhodes et al., Methods Mol. Biol. 55, 121-131, 1995).

Detecting Expression of LALE Polypeptides

Although the presence of marker gene expression suggests that a LALE polynucleotide is also present, its presence and expression may need to be confirmed. For example, if a sequence encoding a LALE polypeptide is inserted within a marker gene sequence, transformed cells containing sequences which encode the LALE polypeptide can be identified by the absence of marker gene function. Alternatively, a marker gene can be placed in tandem with a sequence encoding a LALE polypeptide under the control of a single promoter. Expression of the marker gene in response to induction or selection usually indicates expression of a LALE polynucleotide.

Alternatively, host cells which contain a LALE polynucleotide and which express a LALE polypeptide can be identified by a variety of procedures known to those of skill in the art. These procedures include, but are not limited to, DNA-DNA or DNA-RNA hybridizations and protein bioassay or immunoassay techniques which include membrane, solution, or chip-based technologies for the detection and/or quantification of nucleic acid or protein. For example, the presence of a polynucleotide sequence encoding a LALE polypeptide can be detected by DNA-DNA or DNA-RNA hybridization or amplification using probes or fragments or fragments of polynucleotides encoding the LALE polypeptide. Nucleic acid amplification-based assays involve the use of oligonucleotides selected from sequences encoding the LALE polypeptide to detect transformants which contain a LALE polynucleotide.

A variety of protocols for detecting and measuring the expression of a LALE polypeptide, using either polyclonal or monoclonal antibodies specific for the polypeptide, are known in the art. Examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on a LALE polypeptide can be used, or a competitive binding assay can be employed. These and other assays are described in Hampton et al., SEROLOGICAL METHODS: A LABORATORY MANUAL, APS Press, St. Paul, Minn., 1990) and Maddox et al., J. Exp. Med. 158, 1211-1216, 1983).

A wide variety of labels and conjugation techniques are known by those skilled in the art and can be used in various nucleic acid and amino acid assays. Means for producing labeled hybridization or PCR probes for detecting sequences related to polynucleotides encoding LALE polypeptides include oligolabeling, nick translation, end-labeling, or PCR amplification using a labeled nucleotide. Alternatively, sequences encoding an LALE polypeptide can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, magnetic particles, and the like.

Expression and Purification of LALE Polypeptides

Host cells transformed with nucleotide sequences encoding a LALE polypeptide can be cultured under conditions suitable for the expression and recovery of the protein from cell culture. The polypeptide produced by a transformed cell can be secreted or contained intracellularly depending on the sequence and/or the vector used. As will be understood by those of skill in the art, expression vectors containing polynucleotides which encode LALE polypeptides can be designed to contain signal sequences which direct secretion of LALE polypeptides through a prokaryotic or eukaryotic cell membrane.

As discussed above, other constructions can be used to join a sequence encoding a LALE polypeptide to a nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). Inclusion of cleavable linker sequences such as those specific for Factor Xa or enterokinase (Invitrogen, San Diego, Calif.) between the purification domain and the LALE polypeptide also can be used to facilitate purification. One such expression vector provides for expression of a fusion protein containing a LALE polypeptide and 6 histidine residues preceding a thioredoxin or an enterokinase cleavage site. The histidine residues facilitate purification on IMAC (inmmobilized metal ion affinity chromatography, as described in Porath et al., Prot. Exp. Purif 3, 263-281, 1992), while the enterokinase cleavage site provides a means for purifying the LALE polypeptide from the fusion protein. Vectors which contain fusion proteins are disclosed in Kroll et al., DNA Cell Biol. 12, 441-453, 1993.

Chemical Synthesis

Sequences encoding a LALE polypeptide can be synthesized, in whole or in part, using chemical methods well known in the art (see Caruthers et al., Nucl. Acids Res. Symp. Ser. 215-223, 1980; Horn et al. Nucl. Acids Res. Symp. Ser. 225-232, 1980.) Alternatively, a LALE polypeptide itself can be produced using chemical methods to synthesize its amino acid sequence, such as by direct peptide synthesis using solid-phase techniques (Merrifield, J. Am. Chem. Soc. 85, 2149-2154, 1963; Roberge et al., Science 269, 202-204, 1995). Protein synthesis can be performed using manual techniques or by automation. Automated synthesis can be achieved, for example, using Applied Biosystems 431A Peptide Synthesizer (Perkin Elmer). Optionally, fragments of LALE polypeptides can be separately synthesized and combined using chemical methods to produce a full-length molecule.

The newly synthesized peptide can be substantially purified by preparative high performance liquid chromatography (e.g., Creighton, PROTEINS: STRUCTURES AND MOLECULAR PRINCIPLES, W H Freeman and Co., New York, N.Y., 1983). The composition of a synthetic LALE polypeptide can be confirmed by amino acid analysis or sequencing (e.g., the Edman degradation procedure; see Creighton, supra). Additionally, any portion of the amino acid sequence of the LALE polypeptide can be altered during direct synthesis and/or combined using chemical methods with sequences from other proteins to produce a variant polypeptide or a fusion protein.

Production of Altered LALE Polypeptides

As will be understood by those of skill in the art, it may be advantageous to produce LALE polypeptide-encoding nucleotide sequences possessing non-naturally occurring codons. For example, codons preferred by a particular prokaryotic or eukaryotic host can be selected to increase the rate of protein expression or to produce an RNA transcript having desirable properties, such as a half-life which is longer than that of a transcript generated from the naturally occurring sequence.

The nucleotide sequences disclosed herein can be engineered using methods generally known in the art to alter LALE polypeptide-encoding sequences for a variety of reasons, including but not limited to, alterations which modify the cloning, processing, and/or expression of the polypeptide or mRNA product. DNA shuffling by random fragmentation and PCR reassembly of gene fragments and synthetic oligonucleotides can be used to engineer the nucleotide sequences. For example, site-directed mutagenesis can be used to insert new restriction sites, alter glycosylation patterns, change codon preference, produce splice variants, introduce mutations, and so forth.

Antibodies

Any type of antibody known in the art can be generated to bind specifically to an epitope of a LALE polypeptide. “Antibody” as used herein includes intact immuno-globulin molecules, as well as fragments thereof, such as Fab, F(ab′)₂, and Fv, which are capable of binding an epitope of a LALE polypeptide. Typically, at least 6, 8, 10, or 12 contiguous amino acids are required to form an epitope. However, epitopes which involve non-contiguous amino acids may require more, e.g., at least 15, 25, or 50 amino acids.

An antibody which specifically binds to an epitope of a LALE polypeptide can be used therapeutically, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen.

Typically, an antibody which specifically binds to a LALE polypeptide provides a detection signal at least 5-, 10-, or 20-fold higher than a detection signal provided with other proteins when used in an Immunochemical assay. Preferably, antibodies which specifically bind to LALE polypeptides do not detect other proteins in immunochemical assays and can immunoprecipitate a LALE polypeptide from solution.

LALE polypeptides can be used to immunize a mammal, such as a mouse, rat, rabbit, guinea pig, monkey, or human, to produce polyclonal antibodies. If desired, a LALE polypeptide can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a LALE polypeptide can be prepared using any technique which provides for the production of antibody molecules by continuous cell lines in culture. These techniques include, but are not limited to, the hybridoma technique, the human B-cell hybridoma technique, and the EBV-hybridoma technique (Kohler et al., Nature 256, 495-497, 1985; Kozbor et al., J. Immunol. Methoods 81, 31-42, 1985; Cote et al., Proc. Natl. Acad. Sci. 80, 2026-2030, 1983; Cole et al., Mol. Cell Biol. 62, 109-120, 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81, 6851-6855, 1984; Neuberger et al., Nature 312, 604-608, 1984; Takeda et al., Nature 314, 452-454, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions. Alternatively, humanized antibodies can be produced using recombinant methods, as described in GB2188638B. Antibodies which specifically bind to a LALE polypeptide can contain antigen binding sites which are either partially or fully humanized, as disclosed in U.S. Pat. No. 5,565,332.

Alternatively, techniques described for the production of single chain antibodies can be adapted using methods known in the art to produce single chain antibodies which specifically bind to LALE polypeptides. Antibodies with related specificity, but of distinct idiotypic composition, can be generated by chain shuffling from random combinatorial immunoglobin libraries (Burton, Proc. Natl. Acad. Sci. 88, 11120-23, 1991).

Single-chain antibodies also can be constructed using a DNA amplification method, such as PCR, using hybridoma cDNA as a template (Thirion et al., 1996, Eur. J. Cancer Prev. 5, 507-11). Single-chain antibodies can be mono- or bispecific, and can be bivalent or tetravalent. Construction of tetravalent, bispecific single-chain antibodies is taught, for example, in Coloma & Morrison, 1997, Nat. Biotechnol 15, 159-63. Construction of bivalent, bispecific single-chain antibodies is taught in Mallender & Voss, 1994, J. Biol. Chem. 269, 199-206.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., 1995, Int. J. Cancer 61, 497-501; Nicholls et al., 1993, J. Immunol. Meth. 165, 81-91).

Antibodies which specifically bind to LALE polypeptides also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833-3837, 1989; Winter et al., Nature 349, 293-299, 1991).

Other types of antibodies can be constructed and used therapeutically in methods of the invention. For example, chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies according to the invention can be purified by methods well known in the art. For example, antibodies can be affinity purified by passage over a column to which a LALE polypeptide is bound. The bound antibodies can then be eluted from the column using a buffer with a high salt concentration.

Antisense Oligonucleotides

Antisense oligonucleotides are nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of LALE gene products in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters. See Brown, Meth. Mol. Biol. 20, 1-8, 1994; Sonveaux, Meth. Mol. Biol. 26, 1-72, 1994; Uhlmann et al., Chem. Rev. 90, 543-583, 1990.

Modifications of LALE gene expression can be obtained by designing antisense oligonucleotides which will form duplexes to the control, 5′, or regulatory regions of a LALE gene. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (e.g., Gee et al., in Huber & Carr, MOLECULAR AND IMMUNOLOGIC APPROACHES, Futura Publishing Co., Mt. Kisco, N.Y., 1994). An antisense oligonucleotide also can be designed to block translation of mRNA by preventing the transcript from binding to ribosomes.

Precise complementarity is not required for successful complex formation between an antisense oligonucleotide and the complementary sequence of a LALE polynucleotide. Antisense oligonucleotides which comprise, for example, 2, 3, 4, or 5 or more stretches of contiguous nucleotides which are precisely complementary to a LALE polynucleotide, each separated by a stretch of contiguous nucleotides which are not complementary to adjacent LALE nucleotides, can provide sufficient targeting specificity for LALE mRNA. Preferably, each stretch of complementary contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular LALE polynucleotide sequence.

Antisense oligonucleotides can be modified without affecting their ability to hybridize to a LALE polynucleotide. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′, 5′-substituted oligonucleotide in which the 3′ hydroxyl group or the 5′ phosphate group are substituted, also can be employed in a modified antisense oligonucleotide. These modified oligonucleotides can be prepared by methods well known in the art. See, e.g., Agrawal et al., Trends Biotechnol. 10, 152-158, 1992; Uhlmann et al., Chem. Rev. 90, 543-584, 1990; Uhlmann et al., Tetrahedron. Lett. 215, 3539-3542, 1987.

Ribozymes

Ribozymes are RNA molecules with catalytic activity. See, e.g., Cech, Science 236, 1532-1539; 1987; Cech, Ann. Rev. Biochem. 59, 543-568; 1990, Cech, Curr. Opin. Struct. Biol. 2, 605-609; 1992, Couture & Stinchcomb, Trends Genet. 12, 510-515, 1996. Ribozymes can be used to inhibit gene function by cleaving an RNA sequence, as is known in the art (e.g., Haseloff et al., U.S. Pat. No. 5,641,673). The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences.

The coding sequence of a LALE polynucleotide can be used to generate ribozymes which will specifically bind to mRNA transcribed from the LALE polynucleotide. Methods of designing and constructing ribozymes which can cleave RNA molecules in trans in a highly sequence specific manner have been developed and described in the art (see Haseloff et al. Nature 334, 585-591, 1988). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, for example, Gerlach et al., EP 321,201).

Specific ribozyme cleavage sites within a LALE RNA target can be identified by scanning the LALE target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate LALE RNA targets also can be evaluated by testing accessibility to hybridization with complementary oligo-nucleotides using ribonuclease protection assays. The nucleotide sequences shown in SEQ ID NOS: 1, 3, and 5 and their complements provide sources of suitable hybridization region sequences. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease LALE expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or UAS element, and a transcriptional terminator signal, for controlling transcription of Ribozymes in the cells.

As taught in Haseloff et al., U.S. Pat. No. 5,641,673, ribozymes can be engineered so that ribozyme expression will occur in response to factors which induce expression of a target gene. Ribozymes also can be engineered to provide an additional level of regulation, so that destruction of mRNA occurs only when both a ribozyme and a target gene are induced in the cells.

Screening Methods

The invention provides methods for identifying modulators, i.e., candidate or test compounds which bind to LALE polypeptides or polynucleotides and/or have a stimulatory or inhibitory effect on, for example, expression or activity of the LALE polypeptide or polynucleotide, so as to regulate degradation polypeptides.

The invention provides assays for screening test compounds which bind to or modulate the activity of a LALE polypeptide or a LALE polynucleotide. A test compound preferably binds to a LALE polypeptide or polynucleotide. More preferably, a test compound decreases a peptidase activity of a LALE polypeptide or expression of a LALE polynucleotide by at least about 10, preferably about 50, more preferably about 75, 90, or 100% relative to the absence of the test compound.

Test Compounds

Test compounds can be pharmacologic agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries, while the other four approaches are applicable to polypeptide, non-peptide oligomer, or small molecule libraries of compounds. See Lam, Anticancer Drug Des. 12, 145, 1997.

Methods for the synthesis of molecular libraries are well known in the art (see, for example, DeWitt et al., Proc. Natl. Acad. Sci. U.S.A. 90, 6909, 1993; Erb et al. Proc. Natl. Acad. Sci. U.S.A. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061; Gallop et al., J. Med. Chem. 37, 1233, 1994). Libraries of compounds can be presented in solution (see, e.g., Houghten, Biotechniques 13, 412-421, 1992), or on beads (Lam, Nature 354, 82-84, 1991), chips (Fodor, Nature 364, 555-556, 1993), bacteria or spores (Ladner, U.S. Pat. No. 5,223,409), plasmids (Cull et al., Proc. Natl. Acad. Sci. U.S.A. 89, 1865-1869, 1992), or phage (Scott & Smith, Science 249, 386-390, 1990; Devlin, Science 249, 404-406, 1990); Cwirla et al., Proc. Natl. Acad. Sci. 97, 6378-6382, 1990; Felici, J. Mol Biol. 222, 301-310, 1991; and Ladner, U.S. Pat. No. 5,223,409).

High Throughput Screening

Test compounds can be screened for the ability to bind to LALE polypeptides or polynucleotides or to affect LALE activity or LALE gene expression using high throughput screening. Using high throughput screening, many discrete compounds can be tested in parallel so that large numbers of test compounds can be quickly screened. The most widely established techniques utilize 96-well microtiter plates. The wells of the microtiter plates typically require assay volumes that range from 50 to 500 μl. In addition to the plates, many instruments, materials, pipettors, robotics, plate washers, and plate readers are commercially available to fit the 96-well format.

Alternatively, “free format assays,” or assays that have no physical barrier between samples, can be used. For example, an assay using pigment cells (melanocytes) in a simple homogeneous assay for combinatorial peptide libraries is described by Jayawickreme et al., Proc. Natl. Acad. Sci. U.S.A. 19, 1614-18 (1994). The cells are placed under agarose in petri dishes, then beads that carry combinatorial compounds are placed on the surface of the agarose. The combinatorial compounds are partially released the compounds from the beads. Active compounds can be visualized as dark pigment areas because, as the compounds diffuse locally into the gel matrix, the active compounds cause the cells to change colors.

Another example of a free format assay is described by Chelsky, “Strategies for Screening Combinatorial Libraries: Novel and Traditional Approaches,” reported at the First Annual Conference of The Society for Biomolecular Screening in Philadelphia, Pa. (Nov. 7-10, 1995). Chelsky placed a simple homogenous enzyme assay for carbonic anhydrase inside an agarose gel such that the enzyme in the gel would cause a color change throughout the gel. Thereafter, beads carrying combinatorial compounds via a photolinker were placed inside the gel and the compounds were partially released by UV-light. Compounds that inhibited the enzyme were observed as local zones of inhibition having less color change.

Yet another example is described by Salmon et al., Molecular Diversity 2, 57-63 (1996). In this example, combinatorial libraries were screened for compounds that had cytotoxic effects on cancer cells growing in agar.

Another high throughput screening method is described in Beutel et al., U.S. Pat. No. 5,976,813. In this method, test samples are placed in a porous matrix. One or more assay components are then placed within, on top of, or at the bottom of a matrix such as a gel, a plastic sheet, a filter, or other form of easily manipulated solid support. When samples are introduced to the porous matrix they diffuse sufficiently slowly such that the assays can be performed without the test samples running together.

Binding Assays

For binding assays, the test compound is preferably a small molecule which binds to and occupies the active site of a LALE polypeptide, thereby making the active site inaccessible to substrate such that normal biological activity is prevented. Examples of such small molecules include, but are not limited to, small peptides or peptide-like molecules. In binding assays, either the test compound or the LALE polypeptide can comprise a detectable label, such as a fluorescent, radioisotopic, chemiluminescent, or enzymatic label, such as horseradish peroxidase, alkaline phosphatase, or luciferase. Detection of a test compound which is bound to the LALE polypeptide can then be accomplished, for example, by direct counting of radioemmission, by scintillation counting, or by determining conversion of an appropriate substrate to a detectable product.

Alternatively, binding of a test compound to a LALE polypeptide can be determined without labeling either of the interactants. For example, a microphysiometer can be used to detect binding of a test compound with a LALE polypeptide. A microphysiometer (e.g., Cytosensor™) is an analytical instrument that measures the rate at which a cell acidifies its environment using a light-addressable potentiometric sensor (LAPS). Changes in this acidification rate can be used as an indicator of the interaction between a test compound and a LALE polypeptide. (McConnell et al., Science 257, 1906-1912, 1992).

Determining the ability of a test compound to bind to a LALE polypeptide also can be accomplished using a technology such as real-time Bimolecular Interaction Analysis (BIA) (Sjolander & Urbaniczky, Anal. Chem. 63, 2338-2345, 1991, and Szabo et al., Curr. Opin. Struct. Biol. 5, 699-705, 1995). BIA is a technology for studying biospecific interactions in real time, without labeling any of the interactants (e.g., BIAcore™). Changes in the optical phenomenon surface plasmon resonance (SPR) can be used as an indication of real-time reactions between biological molecules.

In yet another aspect of the invention, a LALE polypeptide can be used as a “bait protein” in a two-hybrid assay or three-hybrid assay (see, e.g., U.S. Pat. No. 5,283,317; Zervos et al., Cell 72, 223-232, 1993; Madura et al., J. Biol. Chem. 268, 12046-12054, 1993; Bartel et al., Biotechniques 14, 920-924, 1993; Iwabuchi et al., Oncogene 8, 1693-1696, 1993; and Brent W094/10300) to identify other proteins which bind to or interact with the LALE polypeptide and modulate its activity.

The two-hybrid system is based on the modular nature of most transcription factors, which consist of separable DNA-binding and activation domains. Briefly, the assay utilizes two different DNA constructs. For example, in one construct a polynucleotide encoding a LALE polypeptide can be fused to a polynucleotide encoding the DNA binding domain of a known transcription factor (e.g., GAL-4). In the other construct a DNA sequence that encodes an unidentified protein (“prey” or “sample”) can be fused to a polynucleotide that codes for the activation domain of the known transcription factor. If the “bait” and the “prey” proteins are able to interact in vivo to form a protein-dependent complex, the DNA-binding and activation domains of the transcription factor are brought into close proximity. This proximity allows transcription of a reporter gene (e.g., LacZ), which is operably linked to a transcriptional regulatory site responsive to the transcription factor. Expression of the reporter gene can be detected, and cell colonies containing the functional transcription factor can be isolated and used to obtain the DNA sequence encoding the protein which interacts with the LALE polypeptide.

It may be desirable to immobilize either a LALE polypeptide (or polynucleotide) or the test compound to facilitate separation of bound from unbound forms of one or both of the interactants, as well as to accommodate automation of the assay. Thus, either the LALE polypeptide (or polynucleotide) or the test compound can be bound to a solid support. Suitable solid supports include, but are not limited to, glass or plastic slides, tissue culture plates, microtiter wells, tubes, silicon chips, or particles such as beads (including, but not limited to, latex, polystirene, or glass beads). Any method known in the art can be used to attach the LALE polypeptide (or polynucleotide) or test compound to a solid support, including use of covalent and non-covalent linkages, passive absorption, or pairs of binding moieties attached respectively to the polypeptide or test compound and the solid support. Test compounds are preferably bound to the solid support in an array, so that the location of individual test compounds can be tracked. Binding of a test compound to a LALE polypeptide (or polynucleotide) can be accomplished in any vessel suitable for containing the reactants. Examples of such vessels include microtiter plates, test tubes, and microcentrifuge tubes.

In one embodiment, a LALE polypeptide is a fusion protein comprising a domain that allows the LALE polypeptide to be bound to a solid support. For example, glutathione-S-transferase fusion proteins can be adsorbed onto glutathione sepharose beads (Sigma Chemical, St. Louis, Mont.) or glutathione derivatized microtiter plates, which are then combined with the test compound or the test compound and the non-adsorbed LALE polypeptide; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly, as described above. Alternatively, the complexes can be dissociated from the solid support before binding is determined.

Other techniques for immobilizing proteins or polynucleotides on a solid support also can be used in the screening assays of the invention. For example, either a LALE polypeptide (or polynucleotide) or a test compound can be immobilized utilizing conjugation of biotin and streptavidin. Biotinylated LALE polypeptides, polynucleotides, or test compounds can be prepared from biotin-NHS(N-hydroxysucein-imide) using techniques well known in the art (e.g., biotinylation kit, Pierce Chemicals, Rockford, Ill.) and immobilized in the wells of streptavidin-coated 96 well plates (Pierce Chemical). Alternatively, antibodies which specifically bind to a LALE polypeptide, polynucleotides, or a test compound, but which do not interfere with a desired binding site, such as the active site of the LALE polypeptide, can be derivatized to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to a LALE polypeptide or test compound, enzyme-linked assays which rely on detecting a LALE activity of the LALE polypeptide, and SDS gel electrophoresis under non-reducing conditions.

Screening for test compounds which bind to a LALE polypeptide or polynucleotide also can be carried out in an intact cell. Any cell which comprises a LALE polynucleotide or polypeptide can be used in a cell-based assay system. A LALE polynucleotide can be naturally occurring in the cell or can be introduced using techniques such as those described above. Either a primary culture or an established cell line, including neoplastic cell lines such as the colon cancer cell lines HCT116, DLD1, HT29, Caco2, SW837, SW480, and RKO, breast cancer cell lines 21-PT, 21-MT, MDA-468, SK-BR3, and BT-474, the A549 lung cancer cell line, and the H392 glioblastoma cell line, can be used. An intact cell is contacted with a test compound. Binding of the test compound to a LALE polypeptide or polynucleotide is determined as described above, after lysing the cell to release the LALE polypeptide-or polynucleotide-test compound complex.

Peptidase Assays

Test compounds can be tested for the ability to increase or decrease peptidase activity of a LALE polypeptide. Peptidase activity can be measured, for example, of a LALE activity can be measured after contacting either a purified LALE polypeptide, a cell extract, or an intact cell with a test compound. A test compound which decreases LALE activity by at least about 10, preferably about 50, more preferably about 75, 90, or 100% is identified as a potential agent for decreasing polypeptide degradation. A test compound which increases LALE activity by at least about 10, preferably about 50, more preferably about 75, 90, or 100% is identified as a potential agent for increasing polypeptide degradation. Peptidase activity can be assayed, for example, as described in Example 2.

LALE Gene Expression

In another embodiment, test compounds which increase or decrease LALE gene expression are identified. A LALE polynucleotide is contacted with a test compound, and the expression of an RNA or polypeptide product of the LALE polynucleotide is determined. The level of expression of LALE mRNA or polypeptide in the presence of the test compound is compared to the level of expression of LALE mRNA or polypeptide in the absence of the test compound. The test compound can then be identified as a modulator of expression based on this comparison. For example, when expression of LALE mRNA or polypeptide is greater in the presence of the test compound than in its absence, the test compound is identified as a stimulator or enhancer of LALE mRNA or polypeptide expression. Alternatively, when expression of LALE mRNA or polypeptide is less in the presence of the test compound than in its absence, the test compound is identified as an inhibitor of LALE mRNA or polypeptide expression.

The level of LALE mRNA or polypeptide expression in the cells can be determined by methods well known in the art for detecting mRNA or polypeptides. Either qualitative or quantitative methods can be used. The presence of polypeptide products of a LALE polynucleotide can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled ammo acids into a LALE polypeptide.

Such screening can be carried out either in a cell-free assay system or in an intact cell. Any cell which expresses a LALE polynucleotide can be used in a cell-based assay system. The LALE polynucleotide can be naturally occurring in the cell or can be introduced using techniques such as those described above. Either a primary culture or an established cell line, including neoplastic cell lines such as the colon cancer cell lines HCT116, DLD1, HT29, Caco2, SW837, SW480, and RKO, breast cancer cell lines 21-PT, 21-MT, MDA-468, SK-BR3, and BT-474, the A549 lung cancer cell line, and the H392 glioblastoma cell line, can be used.

Pharmaceutical Compositions

The invention also provides pharmaceutical compositions which can be administered to a patient to achieve a therapeutic effect. Pharmaceutical compositions of the invention can comprise, for example, a LALE polypeptide, LALE polynucleotide, antibodies which specifically bind to LALE activity, or mimetics, agonists, antagonists, or inhibitors of LALE activity. The compositions can be administered alone or in combination with at least one other agent, such as stabilizing compound, which can be administered in any sterile, biocompatible pharmaceutical carrier, including, but not limited to, saline, buffered saline, dextrose, and water. The compositions can be administered to a patient alone, or in combination with other agents, drugs or hormones.

In addition to the active ingredients, these pharmaceutical compositions can contain suitable pharmaceutically-acceptable carriers comprising excipients and auxiliaries which facilitate processing of the active compounds into preparations which can be used pharmaceutically. Pharmaceutical compositions of the invention can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxy-propylmethyl-cellulose, or sodium carboxymethylcellulose; gums including arabic and tragacanth; and proteins such as gelatin and collagen. If desired, disintegrating or solubilizing agents can be added, such as the cross-linked polyvinyl pyrrolidone, agar, alginic acid, or a salt thereof, such as sodium alginate.

Dragee cores can be used in conjunction with suitable coatings, such as concentrated sugar solutions, which also can contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty-oils, liquid, or liquid polyethylene glycol with or without stabilizers.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers also can be used for delivery. Optionally, the suspension also can contain suitable stabilizers or agents which increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention can be manufactured in a manner that is known in the art, e.g., by means of conventional mixing, dissolving, granulating, dragee-making, levigating, emulsifying, encapsulating, entrapping, or lyophilizing processes. The pharmaceutical composition can be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation can be a lyophilized powder which can contain any or all of the following: 1-50 mM histidine, 0.1%-2% sucrose, and 2-7% mannitol, at a pH range of 4.5 to 5.5, that is combined with buffer prior to use.

Further details on techniques for formulation and administration can be found in the latest edition of REMINGTON'S PHARMACEUTICAL SCIENCES (Maack Publishing Co., Easton, Pa.). After pharmaceutical compositions have been prepared, they can be placed in an appropriate container and labeled for treatment of an indicated condition. Such labeling would include amount, frequency, and method of administration.

Therapeutic Indications and Methods

1. Tumor Growth and Metastases

Elevated levels of serum leucine aminopeptidase have been found in patients with cancer. Garg et al. J. Laryngol. Otol. 108, 660-2, 1994; Aziz et al. J. Indian Med. Assoc. 88, 160-3, 1990; Gupta et al. J. Indian Med. Assoc. 87, 68-70, 1989. Serum leucine aminopeptidase activity increases with increasing lymph node spread. Garg et al. (1994). After treatment for cancer, levels of leucine aminopeptidase are reduced. The human LALE gene therefore provides a therapeutic target for decreasing cellular and extracellular polypeptide degradation, in particular for treating or preventing tumor metastases. Cancers whose growth can be suppressed according to the invention include adenocarcinoma, melanoma, cancers of the adrenal gland, bladder, blood, bone, breast, cervix, colon, gall bladder, head and neck, intestine, liver, lung, ovary, pancreas, prostate, stomach, testis, and uterus.

2. Autoimmune diseases

Inhibitors of LALE can be used to treat or prevent autoimmune diseases such as systemic lupus erythematosus (SLE). Elevated levels of serum leucine aminopeptidase occur in patients with SLE. Inokuma et al. Rheumatology (Oxford) 38, 705-8, 1999. Suppression of LALE activity therefore can be used to treat or prevent autoimmune disease.

3. HIV

Inhibitors of LALE can be used to treat or prevent HIV infection. Elevated levels of serum leucine aminopeptidase have been demonstrated in HIV positive patients. Pulido-Cejudo et al. Antiviral Res. 36, 167-177, 1997. Treatment of cells with an inhibitor of leucine aminopeptidase inhibits cellular and extracelluar activity of leucine aminopeptidase and hinders HIV infection of the cells. Pulido-Cejudo et al. 1997. Suppression of LALE activity therefore can be used to treat or prevent HIV infection.

4. Elevated Blood Pressure

LALE can be used to decrease high blood pressure. The administration of leucine aminopeptidase has been shown to reduce blood pressure in hypertensive rats. Wright et al. Hypertens. 13, 910-5, 1989; Wright et al. J. Hypertens. 8, 969-74, 1990. Therefore, the LALE can be used to treat or prevent high blood pressure.

This invention further pertains to the use of novel agents identified by the screening assays described above. Accordingly, it is within the scope of this invention to use a test compound identified as described herein in an appropriate animal model. For example, an agent identified as described herein (e.g., a modulating agent, an antisense nucleic acid molecule, a specific antibody, ribozyme, or a polypeptide-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

A reagent which affects LALE activity can be administered to a human cell, either in vitro or in vivo, to reduce LALE activity. The reagent preferably binds to an expression product of a LALE gene. If the expression product is a polypeptide, for example, the reagent can be an antibody or a small chemical compound. For treatment of human cells ex vivo, a reagent can be added to a preparation of stem cells which have been removed from the body. The cells can then be replaced in the same or another human body, with or without clonal propagation, as is known in the art.

In one embodiment, the reagent is delivered using a liposome. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent, particularly a polynucleotide, to a particular site in an animal, such as a human. Preferably, the lipid composition of the liposome is capable of targeting to a specific organ of an animal, such as the lung or liver.

A liposome useful in the present invention comprises a lipid composition that is capable of fusing with the plasma membrane of the targeted cell to deliver its contents to the cell. Preferably, the transfection efficiency of a liposome is about 0.5 μg of DNA per 16 nmole of liposome delivered to about 10⁶ cells, more preferably about 1.0 μg of DNA per 16 nmol of liposome delivered to about 10⁶ cells, and even more preferably about 2.0 μg of DNA per 16 nmol of liposome delivered to about 10⁶ cells. Preferably, a liposome is between about 100 and 500 nm, more preferably between about 150 and 450 nm, and even more preferably between about 200 and 400 nm in diameter.

Suitable liposomes for use in the present invention include those liposomes standardly used in, for example, gene delivery methods known to those of skill in the art. More preferred liposomes include liposomes having a polycationic lipid composition and/or liposomes having a cholesterol backbone conjugated to polyethylene glycol. Optionally, a liposome comprises a compound capable of targeting the liposome to a tumor cell, such as a tumor cell ligand exposed on the outer surface of the liposome.

Complexing a liposome with a reagent such as an antisense oligonucleotide or ribozyme can be achieved using methods which are standard in the art (see, for example, U.S. Pat. No. 5,705,151). Preferably, from about 0.1 μg to about 10 μg of polynucleotide is combined with about 8 nmol of liposomes, more preferably from about 0.5 μg to about 5 μg of polynucleotides are combined with about 8 nmol liposomes, and even more preferably about 1.0 μg of polynucleotides is combined with about 8 nmol liposomes.

In another embodiment, antibodies can be delivered to specific tissues in vivo using receptor-mediated targeted delivery. Receptor-mediated DNA delivery techniques are taught in, for example, Findeis et al. Trends in Biotechnol. 11, 202-05 (1993); Chiou et al., GENE THERAPEUTICS: METHODS AND APPLICATIONS OF DIRECT GENE TRANSFER (J. A. Wolff, ed.) (1994); Wu & Wu, J. Biol. Chem. 263, 621-24 (1988); Wu et al., J. Biol. Chem. 269, 542-46 (1994); Zenke et al., Proc. Natl. Acad. Sci. U.S.A. 87, 3655-59 (1990); Wu et al., J. Biol. Chem. 266, 338-42 (1991).

If the reagent is a single-chain antibody, a polynucleotide encoding the antibody can be constructed and introduced into a cell either ex vivo or in vivo using well-established techniques including, but not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and DEAE- or calcium phosphate-mediated transfection.

Effective in vivo dosages of an antibody are in the range of about 5 μg to about 50 μg/kg, about 50 μg to about 5 μg/kg, about 100 μg to about 500 μg/kg of patient body weight, and about 200 to about 250 μg/kg of patient body weight. For administration of polynucleotides encoding single-chain antibodies, effective in vivo dosages are in the range of about 100 ng to about 200 ng, 500 ng to about 50 mg, about 1 μg to about 2 mg, about 5 μg to about 500 μg, and about 20 μg to about 100 μg of DNA.

If the expression product is mRNA, the reagent is preferably an antisense oligonucleotide or a ribozyme. Polynucleotides which express antisense oligonucleotides or ribozymes can be introduced into cells by a variety of methods, as described above.

Preferably, a reagent reduces expression of a LALE gene or the activity of a LALE polypeptide by at least about 10, preferably about 50, more preferably about 75, 90, or 100% relative to the absence of the reagent. The effectiveness of the mechanism chosen to decrease the level of expression of a LALE gene or the activity of a LALE polypeptide can be assessed using methods well known in the art, such as hybridization of nucleotide probes to LALE-specific mRNA, quantitative RT-PCR, immunologic detection of a LALE polypeptide, or measurement of LALE activity.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects.

Determination of a Therapeutically Effective Dose

Determination of a therapeutically effective dose is well within the capability of those skilled in the art. A therapeutically effective dose refers to that amount of active ingredient which increases or decreases LALE activity relative to LALE activity which occurs in the absence of the therapeutically effective dose.

For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀.

Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Normal dosage amounts of any particular reagent can vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for polypeptides or their inhibitors. Similarly, delivery of polynucleotides or polypeptides will be specific to particular cells, conditions, locations, etc.

Any of the therapeutic methods described above can be applied to any subject in need of such therapy, including, for example, mammals such as dogs, cats, cows, horses, rabbits, monkeys, and most preferably, humans.

The above disclosure generally describes the present invention, and all patents and patent applications cited in this disclosure are expressly incorporated herein. A more complete understanding can be obtained by reference to the following specific examples which are provided for purposes of illustration only and are not intended to limit the scope of the invention.

### EXAMPLE 1

Detection of LALE Activity

The polynucleotide of SEQ ID NO: 7 is inserted into the expression vector pCEV4 and the expression vector pCEV4-LALE polypeptide obtained is transfected into human embryonic kidney 293 cells.

LALE activity is analyzed using fluorogenic substrates of the amino acid-AMC type (Bachem, King of Prussia, Pa.). Substrates containing 19 different N-terminal amino acids (except tryptophan) are used at a concentration of 200 μM of each in a 1-ml volume of 50 mM tris-HCl, 5 mM MgCl12, pH 8.5. Substrate hydrolysis is determined using 10 μg of cytosolic proteins from the transfected cells mentioned above, as measured using the Coomassie kit from Pierce (Rockford, Ill.). Samples are incubated for 75 min at 37° C., and the reaction is stopped by adding 1 μl of 10% SDS, and then fluorescence is measured at an excitation wavelength of 380 nm and an emission wavelength of 440 nm in a SLM-AMINCO spectrometer (Rochester, N.Y.). Analysis of fractionated extracts is carried out with 50 μl of each fraction in 500 μl of 50 mM Tris-HCl, 5 mM MgC12, ph 8.5, stopped after 75 min incubation at 37° C. with 500 ml of 2% SDS, and analyzed as described above. The LALE activity of the polypeptide with the amino acid sequence of SEQ ID NO: 8 is shown.

### EXAMPLE 2

Identification of a Test Compound Which Binds to a LALE Polypeptide

Purified LALE polypeptides comprising a glutathione-S-transferase protein and absorbed onto glutathione-derivatized wells of 96-well microtiter plates are contacted with test compounds from a small molecule library at pH 7.0 in a physiological buffer solution. LALE polypeptides comprise an amino acid sequence shown in SEQ ID NOS: 2, 4, or 6. The test compounds comprise a fluorescent tag. The samples are incubated for 5 minutes to one hour. Control samples are incubated in the absence of a test compound.

The buffer solution containing the test compounds is washed from the wells. Binding of a test compound to a LALE polypeptide is detected by fluorescence measurements of the contents of the wells. A test compound which increases the fluorescence in a well by at least 15% relative to fluorescence of a well in which a test compound was not incubated is identified as a compound which binds to a LALE polypeptide.

### EXAMPLE 3

Identification of a Test Compound Which Decreases LALE Activity

Cellular and extracellular extracts from the human colon cancer cell line HCT116 are contacted with test compounds from a small molecule library and assayed for-LALE activity. Control extracts in the absence of a test compound also are assayed.

LALE activity is determined fluorometrically using leucine-μ-naphthylamide as the substrate. Kuramochi et al. J. Antibiot. 40, 1605-1611, 1987. The reaction is stopped by boiling the samples at 100° C. for 10 minutes, followed by centrifugation at 780×g at 4° C. for 10 min. A test compound which decreases LALE activity of the extract relative to the control extract by at least 20% is identified as a LALE inhibitor.

### EXAMPLE 4

Identification of a Test Compound Which Decreases LALE Gene Expression

A test compound is administered to a culture of the breast tumor cell line MDA-468 and incubated at 37° C. for 10 to 45 minutes. A culture of the same type of cells incubated for the same time without the test compound provides a negative control.

RNA is isolated from the two cultures as described in Chirgwin et al., Biochem. 18, 5294-99, 1979. Northern blots are prepared using 20 to 30 μg total RNA and hybridized with a ³²P-labeled LALE-specific probe at 65° C. in Express-hyb (CLONTECH). The probe comprises at least 11 contiguous nucleotides selected from SEQ ID NOS: 1, 3, or 5. A test compound which decreases the LALE-specific signal relative to the signal obtained in the absence of the test compound is identified as an inhibitor of LALE gene expression.

### EXAMPLE 5

Treatment of a Breast Tumor with a Reagent which Specifically Binds to a LALE Gene Product

Synthesis of antisense LALE oligonucleotides comprising at least 11 contiguous nucleotides selected from SEQ ID NOS: 1, 3, or 5 is performed on a Pharmacia Gene Assembler series synthesizer using the phosphoramidite procedure (Uhlmann et al., Chem. Rev. 90, 534-83, 1990). Following assembly and deprotection, oligonucleotides are ethanol-precipitated twice, dried, and suspended in phosphate-buffered saline (PBS) at the desired concentration. Purity of these oligonucleotides is tested by capillary gel electrophoreses and ion exchange HPLC. Endotoxin levels in the oligonucleotide preparation are determined using the Limulus Amebocyte Assay (Bang, Biol. Bull. (Woods Hole, Mass.) 105, 361-362, 1953).

An aqueous composition containing the antisense oligonucleotides at a concentration of 0.1-100 μM is injected directly into a breast tumor with a needle. The needle is placed in the tumors and withdrawn while expressing the aqueous composition within the tumor.

The breast tumor is monitored over a period of days or weeks. Additional injections of the antisense oligonucleotides can be given during that time. Growth of the breast tumor is suppressed due to decreased LALE activity of the breast tumor cells.

### EXAMPLE 6

Treatment of High Blood Pressure with a LALE Polypeptide

LALE polypeptides comprising at least 6 amino acids from the sequences selected from SEQ ID NOS: 2, 4, or 6 are infused by i.v. into humans suffering from high blood pressure. Blood pressure is monitored. Blood pressure is decreased due to LALE activity.

1. An isolated polynucleotide encoding a LALE polypeptide and being selected from the group consisting of:
a) a polynucleotide encoding a LALE polypeptide comprising an amino acid sequence selected from the group consisting of:
amino acid sequences which are at least about 50% identical to the amino acid sequence shown in SEQ ID NO: 2;
the amino acid sequence shown in SEQ ID NO: 2;
amino acid sequences which are at least about 50% identical to the amino acid sequence shown in SEQ ID NO: 4;
the amino acid sequence shown in SEQ ID NO: 4;
amino acid sequences which are at least about 50% identical to the amino acid sequence shown in SEQ ID NO: 6;
the amino acid sequence shown in SEQ ID NO: 6;
amino acid sequences which are at least about 50% identical to the amino acid sequence shown in SEQ ID NO: 8; and
the amino acid sequence shown in SEQ ID NO: 8.
b) a polynucleotide comprising the sequence of SEQ ID NOS: 1, 3, 5 or 7;
c) a polynucleotide which hybridizes under stringent conditions to a polynucleotide specified in (a) and (b);
d) a polynucleotide the sequence of which deviates from the polynucleotide sequences specified in (a) to (c) due to the degeneration of the genetic code; and
e) a polynucleotide which represents a fragment, derivative or allelic variation of a polynucleotide sequence specified in (a) to (d).

2. An expression vector containing any polynucleotide of claim 1.

3. A host cell containing the expression vector of claim 2.

4. A substantially purified LALE polypeptide encoded by a polynucleotide of claim 1.

5. A method for producing a LALE polypeptide, wherein the method comprises the following steps:
a) culturing the host cell of claim 3 under conditions suitable for the expression of the LALE polypeptide; and
b) recovering the LALE polypeptide from the host cell culture.

6. A method for detection of a polynucleotide encoding a LALE polypeptide in a biological sample comprising the following steps:
a) hybridizing any polynucleotide of claim 1 to a nucleic acid material of a biological sample, thereby forming a hybridization complex; and
b) detecting said hybridization complex.

7. The method of claim 6, wherein before hybridization, the nucleic acid material of the biological sample is amplified.

8. A method for the detection of a polynucleotide of claim 1 or a LALE polypeptide of claim 5 comprising the steps of contacting a biological sample with a reagent which specifically interacts with the polynucleotide or the LALE polypeptide.

9. A diagnostic kit for conducting the method of any one of claims 6 to 8.

10. A method of screening for agents which decrease the activity of a LALE, comprising the steps of:
contacting a test compound with any LALE polypeptide encoded by any polynucleotide of claim 1;
detecting binding of the test compound of the LALE polypeptide, wherein a test compound which binds to the polypeptide is identified as a potential therapeutic agent for decreasing the activity of a LALE.

11. A method of screening for agents which regulate the activity of a LALE, comprising the steps of:
contacting a test compound with a LALE polypeptide encoded by any polynucleotide of claim 1; and
detecting a LALE activity of the polypeptide, wherein a test compound which increases the LALE activity is identified as a potential therapeutic agent for increasing the activity of the LALE, and wherein a test compound which decreases the LALE activity of the polypeptide is identified as a potential therapeutic agent for decreasing the activity of the LALE.

12. A method of screening for agents which decrease the activity of a LALE, comprising the steps of:
contacting a test compound with any polynucleotide of claim 1 and detecting binding of the test compound to the polynucleotide, wherein a test compound which binds to the polynucleotide is identified as a potential therapeutic agent for decreasing the activity of LALE.

13. A method of reducing the activity of LALE, comprising the steps of:
contacting a cell with a reagent which specifically binds to any polynucleotide of claim 1 or any LALE polypeptide of claim 4, whereby the activity of LALE is reduced.

14. A reagent that modulates the activity of a LALE polypeptide or a polynucleotide wherein said reagent is identified by the method of any of the claims 10 to 12.

15. A pharmaceutical composition, comprising:
the expression vector of claim 2 or the reagent of claim 14 and a pharmaceutically acceptable carrier.

16. Use of the pharmaceutical composition of claim 15 for modulating the activity of a LALE in a disease.

17. Use of claim 16, wherein the disease is cancer, HIV, autoimmune disease, and high blood pressure.

18. Use of the pharmaceutical composition of claim 15 for modulating the activity of a LALE to regulate cellular and extracellular polypeptide degradation.
